(19) United States
(12) Patent Application Publication
(10) Pub. No.: US 2004/0009955 A1
(43) Pub. Date: Jan. 15, 2004

(54) Method of prophylaxis

(76) Inventors: Roy H. Larsen, Bekkestua (NO); Oyvind S. Bruland, Hosle (NO)

Correspondence Address: CLARK & ELBING LLP, 101 FEDERAL STREET, BOSTON, MA, 02110, US

(21) Appl. No.: 10/449,205

(22) Filed: May 30, 2003

(30) Foreign Application Priority Data
Jun. 10, 2002 (GB) GB 0213261.1

(51) Int. Cl. A61K 51/00; A61K 31/66

(52) U.S. Cl. 514/102; 424/1.11

(57) ABSTRACT

A method of prophylaxis of a subject having or having had a soft tissue cancer, whereby to combat metastatic bone cancer therein, the method comprising administering to said subject a physiologically tolerable polyphosphonate and, parenterally, a prophylactically effective amount of a soluble radium-223 or -224 salt or complex. A kit comprising a first container containing a soluble radium-223 or -224 salt or complex and a second container containing at least one dose of a physiologically tolerable polyphosphonate is also provided.

### CROSS REFERENCE TO RELATED APPLICATIONS

This application claims the benefit of British Application No. GB 0213261.1, filed Jun. 10, 2002, the specification of which is hereby incorporated by reference.

### FIELD OF THE INVENTION

The present invention relates to the prophylactic treatment of metastatic malignant diseases at bone surfaces and particularly to methods and medicaments for the prophylactic treatment of metastatic bone cancers by combination treatment.

### BACKGROUND TO THE INVENTION

Normal healthy bone is a dynamic organ, undergoing continuous remodelling throughout the lifetime of the organism by a process of coupled bone resorption and new bone formation. This process occurs at discrete sites and serves to repair and renew the bone and thus maintain skeletal integrity.

In a healthy cycle of bone remodelling, osteoclasts form at the bone surface, where they serve to demineralise a small region of bone and degrade the bone matrix. Osteoblast precursors are then attracted to the remodelling site where they proliferate and mature, forming new bone to replace the material removed. Skeletal equilibrium is maintained by a complex system of local and systemic factors.

One of the causes of morbidity and mortality in patients suffering from certain malignant diseases of soft tissue is the formation of skeletal metastases. These are the result of the migration of malignant cells to the bone surface where they may serve to disrupt the normal cycle of bone remodelling by the misregulation of osteoclastic or osteoblastic activity, the acceleration of bone turnover or the uncoupling of the bone resorption and bone formation events.

Skeletal metastases are associated with high levels of bone pain, hypercalcemia and an increased risk of fracture. Debilitating bone pain in the spine, chest, shoulder or hip is not uncommon due to lesions, caused by the unregulated resorption and/or deposition of bone. Vertebral fracture can also result in severe bone pain and occasionally paralysis and may heal poorly due to highly abnormal bone surrounding the fracture site.

In the treatment of primary cancers of soft tissue, while the patient is routinely monitored to determine whether secondary tumours or metastases develop, e.g, in other soft tissues such as the liver, brain, lymph nodes or breast tissue, it has not been normal practice to give any prophylactic treatment against metastases, in particular bone metastases.

### SUMMARY OF THE INVENTION

The present inventors however have surprisingly found that incidence of bone metastases may be reduced or avoided by the prophylactic treatment of patients diagnosed with primary soft tissue cancers with the combination of an α-emitting radium radionuclide (generally radium-223) and a polyphosphonate (generally a bisphosphonate), in effect a “sterilise and seal” treatment of the patient's bone surfaces.

Thus viewed from one aspect the invention provides a method of prophylaxis of a human or non-human animal (e.g. mammalian, avian or reptilian) subject having or having had a soft tissue cancer to combat metastatic bone cancer therein, said method comprising administering to said subject a physiologically tolerable polyphosphonate and, parenterally, a prophylactically effective amount of a soluble radium-223 or -224 salt or complex.

The method of the present invention is a method of prophylaxis rather than therapy, i.e. the subjects are ones for which while primary soft tissue cancers have been detected and may have been treated by surgery, irradiation or chemotherapy, bone metastases have not been detected.

Viewed from a further aspect the invention provides the use of a soluble radium-233 or -224 salt or complex for the manufacture of a medicament for use in a method of prophylaxis of a human or non-human animal (e.g. mammalian, avian or reptilian) subject to having or having had a soft tissue cancer to combat metastatic bone cancer therein, said method comprising administering to said subject a physiologically tolerable polyphosphonate and, parenterally, a prophylactically effective amount of a soluble radium-223 or -224 salt or complex.

Viewed from a still further aspect the Invention provides the use of a physiologically tolerable polyphosphonate for the manufacture of a medicament for use in a method of prophylaxis of a human or non-human animal (e.g. mammalian, avian or reptilian) subject having or having had a soft tissue cancer to combat metastatic bone cancer therein, said method comprising administering to said subject a physiologically tolerable polyphosphonate and, parenterally, a prophylactically effective amount of a soluble radium-223 or -224 salt or complex.

Viewed from a yet still further aspect the invention provides a kit comprising (a) a first container containing a soluble radium-223 or -224 salt or complex and (b) a second container containing a physiologically tolerable polyphosphonate.

### BRIEF DESCRIPTION OF THE DRAWINGS

FIG. 1 represents the survival of tumour bearing rats when treated by radium-223 only.

FIG. 2 represents the survival of tumour bearing rats when treated by a method of the invention.

### DETAILED DESCRIPTION OF THE INVENTION

The soluble radium salt or complex used according to the invention may be any salt or complex capable of allowing radium uptake by the bone (radium, being a calcium analog, is naturally bone-seeking in its ionic form). The counterions or complexants will desirably themselves be physiologically tolerable and suitable such materials are well-known from the field of pharmaceutical chemistry. Examples include halide (especially chloride), and other inorganic or organic acid anions as well as weak complexants. The radium of the invention may be, but is preferably not, administered as a polyphosphonate-bound complex. Desirably the radium in formulated with the soluble salt or complex already in aqueous solution, e.g. in water for injections. The radium-223 of -224 content or the radium salt or complex need not of course be 100% —all that is required is that the radium α-emitter content be sufficient to achieve the desired prophylactic effect.

The combination treatment of the current invention has the advantage that the polyphosphonate potentiates or synergistically enhances the affect of the radium radioisotope in treating metastases at the bone surface. The treatment of the invention may therefore cause a greater prophylactic effect from a particular dose of radionuclide or allow for a lower dosage of radioisotope without compromising prophylactic effectiveness. The myelotoxicity of the treatment may therefore be reduced in comparison with other radionuclide treatments.

The radium may also be administered in a solution containing cations of at least one other alkaline earth metal, particularly calcium.

The radioactive decay series beginning at ²²³Ra and ending at stable ²⁰⁷Pb is given in Table 1. The corresponding main decay chain from ²³⁴Ra to ²⁰⁸Pb is shown in Table 2.

TABLE 1
²²³Ra decay chain

| Nuclide | ²²³Ra | | ²¹⁹Rn | | ²¹⁵Po | | ²¹¹Pb | | ²¹¹Bi | | ²⁰⁷Tl | | ²⁰⁷Pb |
|---|---|---|---|---|---|---|---|---|---|---|---|---|---|
| ½-life | 11.4 | d | 4.0 | s | 1.8 | ms | 36.1 | m | 2.2 | m | 4.8 | m | stable |
| α-energy/ | 5.64 | | 6.75 | | 7.39 | | | | 6.55 | | | | |
| MeV | | | | | | | | | | | | | |
| β-energy | | | | | | | 1.37 | | | | 1.42 | | |
| (max)/MeV | | | | | | | | | | | | | |
| Energy % | 20.7 | | 24.8 | | 27.1 | | 1.7 | | 24 | | 1.7 | | |

It can be seen from Tables 1 and 2 that both radium nuclei deposit a high proportion of their emitted energy in the form of four high-energy α-particles and only a small fraction (3% in the case of ²²³Ra) as β-emissions. If all daughter nuclei are retained at the bone surface, then these decay chains provide a very high dose of high-LET, short range radiation with only a small proportion of the energy being released as penetrating β-emissions.

TABLE 2
²²⁴Ra decay chain

| Nuclide | ²²⁴Ra | | ²²⁰Rn | | ²¹⁶Po | | ²¹²Pb | | ²¹²Bi* | | ²¹²Po | | ²⁰⁸Pb |
|---|---|---|---|---|---|---|---|---|---|---|---|---|---|
| ½-life | 3.6 | d | 55.6 | s | 0.15 | s | 10.6 | h | 1.0 | h | 0.3 | μs | stable |
| α-energy/ | 5.69 | | 6.29 | | 6.78 | | | | | | 8.78 | | |
| MeV | | | | | | | | | | | | | |
| β-energy | | | | | | | 0.57 | | 2.25 | | | | |
| (max)/MeV | | | | | | | | | | | | | |
| Energy % | 18.8 | | 20.8 | | 22.5 | | 1.3 | | 7.5 | | 29.1 | | |

*²¹²Bi has a branched decay with 54% β-decay (as above) and 36% α-decay (6.2 MeV) to ²⁰⁸Tl, which in turn decays to ²⁰⁸Pb (3.1 min, β).

Since the penetration of α-particles in tissue is typically only 40-90 μm, α-emitters may be expected to give highly localised irradiation with only limited damage to the surrounding tissues. The use of radium isotopes, and particularly ²²³Ra in the present invention allows for conditioning of bone surfaces without exposing the red marrow to high levels of ionising radiation. The myelotoxicity of the treatment is thereby reduced, allowing for reduced side effects and allowing for higher doses to be administered without narrow toxicity proving limiting.

In order to provide a high dose or radiation to the bone surface, it is necessary for a high proportion of the radioisotope radium daughter nuclides to be retained in the vicinity of the bone. If this is not the case then not only will the target area receive a lower radiation dose, but there is a greater risk of soft-tissue damage caused by released radioisotopes. This may appear as damage to the bone marrow, intestines, liver or kidneys. ²²³Ra and ²²⁴Ra, having bone-targeting ability and half-lives of several days are particularly suitable because they may be incorporated deeper into the bone before decaying than shorter-lived α-emitters such as ²¹¹At(½-life 7.2 hours) or ²¹²Bi(½-life 1 hour). ²²³Ra, having the longest half-life, is particularly preferable in this respect.

Radium-223 is also preferable because its daughter nuclide, radon-219 has a very short half-life. Radon is an inert gas and might otherwise tend to diffuse away from the bone surface more quickly than the metallic elements in the radium decay chains. It is therefore advantageous that ²¹⁹Rn has a half-life of only a few seconds.

The decay of radium to lead shown in Tables 1 and 2 is also accompanied by the emission of some additional energy in the form of gamma radiation (>0.3 MeV in total for the decay chain ²²³Ra to ²⁰⁷Pb). The absorbed dose from this radiation is relatively low but the presence of characteristic gamma emissions allows any significant redistribution of the radionuclides within the body to be monitored by gamma spectroscopy.

In a preferred embodiment of the invention therefore, the radium nuclide is radium-223.

Radium-223 and radium-224 may be prepared and purified to pharmaceutical standards by methods known in the art. In particular, Henriksen et al. (Radiochem. Acta 89 (2001), 661-666) recently reported a generator of ²²³Ra in which ²²⁷Ac (½-life 21.77 years) was absorbed onto a silica-based, f-block element sensitive, resin and decayed to ²²³Ra via ²²⁷Th (½-life 18.7 days). Radium-224 may be extracted from a purified precipitate of thorium-228 hydroxide using a solution of calcium chloride (Delikan, Health Physics 35 (1978), 21-24). These publications are hereby incorporated by reference. Alternatively, ²²⁴Ra may be extracted from a preparation of ²²⁵Th by the use of an f-block element sensitive resin or an f-block element specific extraction method. The resin and/or f-block element specific moieties described by Henriksen et al. (Radiochem. Acta 89 (2001), 661-666) for the preparation of ²²³Ra may be used for this purpose.

The radium isotopes used according to the invention are administered parenterally, particularly by intravenous injection or infusion. Suitable formulations for this purpose may be prepared using methods well know in the art and may comprise for example pH modifiers such as buffer solutions, salts and other conicity modifiers, water for injection and other pharmaceutically acceptable carriers and excipients.

In addition, particularly suitable formulations may contain additional alkaline earth metal cations, such as calcium, magnesium and/or strontium ions, agents serving to prevent radium precipitation or colloid formation and/or chelating or complexing agents. Where complexing agents are added, these are preferable weak complexing agents such as organic acids (e.g. oxalic, oxaloacetic, tartaric, succinic, malic and/or malonic acids).

The term polyphosphonate as used herein relates to compounds having two or more phosphonate groups, e.g. 2 to 6 groups, more preferably 2 groups

The polyphosphonates for use in the current invention are typically analogues or naturally occurring pyrophosphate, containing at least one P-C bond. This bond mimics the P-O bond in pyrophosphate but is not susceptible to enzymic hydrolysis. Polyphosphonates, and particularly bisphosphonates are known for their inhibitory effect on osteoclastic bone resorption and have been used for the treatment of Osteoporosis, Paget's disease of bone and tumour induced hypercalcemia.

Polyphosphonates useful in the current invention include complexing polyphosphonates, such as ethylene-diamine-tetra(methylene-phosphonic acid) (EDTMP) or 1,4,7,10-tetraazacyclododecane-1,4,7,10-tetra(methylene-phosphonic acid) (DOTMP), alkyl-nitrogen containing bisphosphonates such as pamidronate ([1-hydroxy-3-aminopropylidene]bis-phosphonic acid, disodium salt), aledronate, ([1-hydroxy-4-aminobutylidene]bis-phosphonic acid, monosodium salt) or ibandronate ([1-hydroxy-3-(methylpentyamino)-butylidene]bis-phosphonic acid, monosodium salt), heterocyclic-nitrogen containing bisphosphonates such as zoledronate (2-(imidazol-1-yl-hydroxyethylene-1,1,-bisphosphonic acid) or risedronate ([1-hydroxy-2-(3-pyridyl)-ethylidene]bis-phosphonic acid, monosodium salt) and non-nitrogen containing bisphosphonates such as etidronate ([1-hydroxyethylidene]bis-phosphonic acid, disodium salt) or clodronate ([dichloromethylene]bis-phosphonic acid, disodium salt). Preferably the polyphosphonate is not a complexing polyphosphonate and is not etidronate, due to its tendency to inhibit bone mineralisation. Preferred polyphosphonates are bisphosphonates such as pamidronate, aledronate, ibandronate, zoledronate or risedronate, particularly ibandronate or pamidronate.

Where the polyphosphonate is in ionic form, a physiologically tolerable counter ion will be used, e.g. sodium, ammonium, or quaternary organic amine. Such physiologically tolerable counterions are well known in the pharmaceutical field.

Suitable polyphosphonates are available commercially in many countries and from many suppliers. Examples include pamidronate, which is produced by Novartis under the name of Aredia(RTM) and available in the USA, Canada, Australia, New Zealand, Japan and throughout Europe, aledronate, which is produced by Merck, Sharp & Dohme under the name of Fosamax(RTM) and available in Italy, Denmark, Sweden and Mexico and clodronate, which is produced e.g. by Boehringer and Astra under the name of Bonefos(RTM) or Ostac(RTM) and is available in Canada, Hong Kong, New Zealand, Russia, and most of Europe. Etidronate is also available in most countries of the World from Procter and Gamble under the name Didronel(RTM).

The polyphosphonates used according to the current invention are preferably bisphosphonates but may alternatively be polyphosphonates other than bisphosphonates, such am tri-, tetra- or penta-phosphonates.

The polyphosphonates are preferably administered orally or parenterally, e.g. by injection or infusion, and commercial preparations are available for administration in each of these ways. Oral or parenteral preparations may also be prepared by methods well known in the art, by the combination of the polyphosphonate with suitable carriers and excipients.

The current invention provides a method for the prophylactic treatment of a subject with diagnosed soft-tissue cancer but without any symptoms or clinical manifestations of metastatic bone disease. In particular the current invention provides a method for the prophylactic treatment of a subject with diagnosed breast, lung, kidney, urinary tract or prostate cancer, or with multiple myeloma, most particularly for the treatment of a subject with diagnosed breast or prostate cancer. Alternatively, the subject may at an earlier stage have had previous bone metastases that have been successfully treated e.g. by surgery, external beam therapy or chemotherapy (for example by hormonal therapy) and the treatment according to the invention may be used as a prophylxis against the formation of further bone metastases.

In some circumstances it will be preferable to apply the method of the invention to certain sub-populations of subjects with particular diagnosed soft-tissue cancers. Such sub-populations may be, for example, those with cancer cells expressing high levels of certain receptors or proteins, such as oestrogen receptors, androgen receptors, laminin receptors or the parathyroid hormone relate protein (PTHrP), or those with cancer cells expressing decreased levels of cell-adhesion proteins such as E-cadherin. Combinations of these indicators may be used to identify suitable subject sub-populations. This forms a further embodiment of the invention.

The combination treatment of the invention is achieved by the administration of a radium isotope, preferably ²²³Ra, and a polyphosphonate, particularly a bisphosphonate. To achieve this, the radium may be administered prior or subsequent to the administration of the polyphosphonate, or the two may be administered concurrently. When the two are administered at the same time, they are preferably not administered in the same preparation but may be administered, e.g. by injection or infusion, at separate sites. Alternatively, the polyphosphonate may be administered orally and the radium parenterally. It is preferred that the radium isotope be administered prior to the administration of the polyphosphonate. Where this is the case, the polyphosphonate may be administered within a short period (e.g. within 1 hour) of the administration of the radium, but more preferably it is administered after a period of between 1 hour and 30 days, still more preferably after a period of between 3 hours and 14 days (e.g. between 1 and 7 days), after radium administration.

The administration of radium and polyphosphonate according to the current invention may be effected single doses, or as a sequence of doses of either one or both components. In particular the radium component may be administered as between 1 and 30, particularly between 1 and 10 doses, each containing between 3.7 kBq and 3700 MBq, preferably between 50 kBq and 10 MBq, more preferably 100 kBq to 20 MBq. Such doses will typically be administered at intervals comparable to the half-life of the radium isotope (e.g. at intervals of between 3 and 90 days, particularly between 10 and 30 days). Such doses may each contain equal quantities of radioisotope, or may contain increasing or decreasing quantities of radium.

Polyphosphonate administration may be in a single dose or in multiple doses, and where the radium is administered as multiple doses the polyphosphonate may be administered to correspond with each radium dose, or in more or fewer doses. Preferably, each dose of radium is followed by at least one dose of polyphosphonate. Polyphosphonate may for example be administered daily between doses of radium. Suitable polyphosphonate dosages for use in the current invention are comparable to documented therapeutic doses for bisphosphonates known in the art. Suitable amounts range from between 1 mg and 5 g (eg. 10 mg and 5 g), particularly between 5 mg and 3, 5 g (e.g 30 mg and 3.5 g) for both oral and parenteral administration. The standard dose of Zometa, for example is 5 mg). The kit of the invention thus preferably comprises suitable doses of radium and polyphosphonate, e.g. for a single day's treatment or for a single radium dose.

The kit may optionally include instructions and/or means to encourage adherence to a pre-defined treatment regime and/or administration means, e.g. syringes (for example pre-filled syringes), giving sets, catheters, venfloms, etc.

The invention is illustrated by the following non-limiting examples and by the attached figures in which;

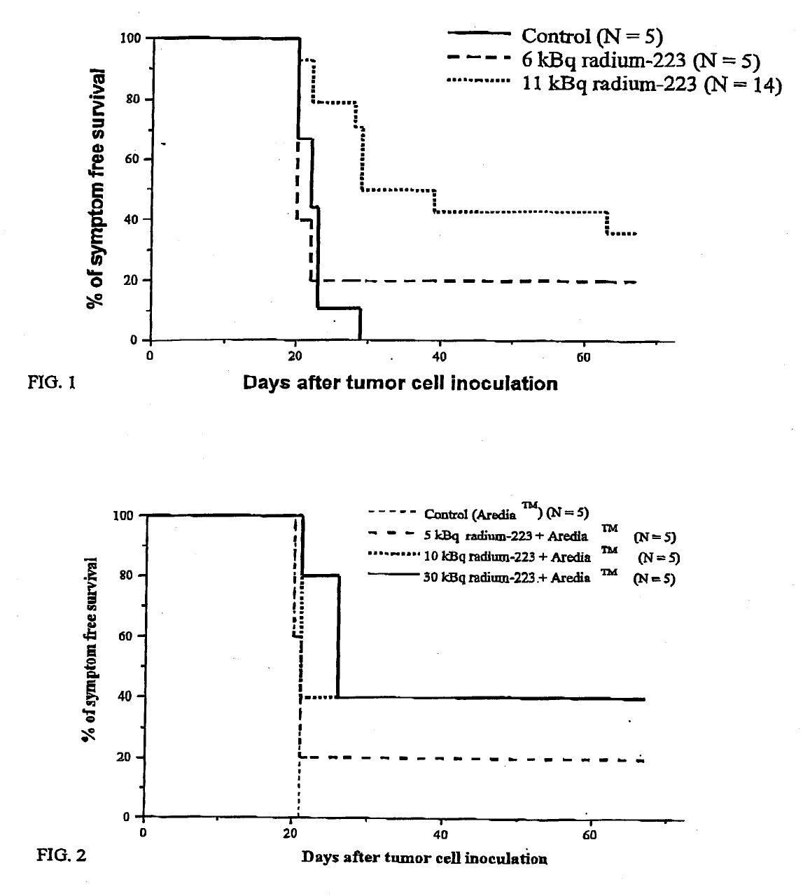

FIG. 1 Shows the effect of 223-radium only in prolonging symptom-free survival in nude rats.

FIG. 2 Shows the effect of 223-radium and the bisphosphonate Aredia in prolonging symptom-free survival in nude rats.

### Example 1

### Radionuclide Production

²²³Ra was produced using a ²²⁷Ac based generator system as described by Henriksen et al. (radiochim Act 89 (2001), 661-666 ). Briefly, ²²⁷Ac and ²²⁷Th were immobilized on the actinide-selective extraction chromatographic material Dipex-2 (EiChrom, Darien Ill.) which is based on P,P′-di(2-ethylhexyl) methanediphosphonic acid. After elution of ²²³Ra from the generator in 1 M HNO₃; ²²³Ra was concentrated on a column of 3 mm i.d. and length of 20 mm, containing 0.1 g of AG 50W-X12 cation exchange resin (Bio-Rad, Hercules, Calif.) at a flow rate of 2 ml/cm².min. Thereafter, the column was washed with 10 ml 1 M HNO₃. ²²³Ra was stripped from the column with 2-3 ml 8 M HNO₃.

The eluate containing ²²³Ra was evaporated to dryness and thereafter 5 mM sodium citrate, pH 7.4, was added followed by filtration through sterile 0.22 μm nylon filters (Nalgene, Rochester, N.Y.).

The final solution used for injection had a ²²³Ra activity concentration or 25-150 kBq/ml.

### Example 2

### Animal Study

Soft tissue tumors were established in nude rats (Han: rnu/rnu Rowett) by intraventricular injection of approximately 1.10⁶ MT-1 breast cancer cells into the left side of the heart (see Engebraaten et al. Int J Cancer 83, (1999) 219-225).

The effect of ²²³Ra in prolonging symptom free survival of rate was studied for two treatment regimens [A] comparative and [B] according to the invention.

Treatment regimen [A]: Animals were treated seven days after tumor cell inoculation. At this time animal body weights varied between 80 and 110 grams and animals were randomized in groups so the average weight for a group was about 100 g at the time of treatment. The treatment groups received 11 or 30 kBq of ²²³Ra administered by tail vein injection while animals in the control group were injected with an equivalent volume of 5 mM sodium citrate solution.

Treatment regimen [B]: 3-Amino-1-hydroxypropylidene bisphosphonate, di-sodium salt (Aredia™, Novartis AG, Switzerland), was added as a supplemental treatment to ²²³Ra. Animals in the treatment groups were treated on day 7 after tumor cell inoculation with 10 or 30 kBq of ²²³Ra. The following day, 1.5 mg/kg b.w. of Aredia™, was administered by i.v. injection. Animals in the control group received 5 mM sodium citrate solution on day 7 and 1.5 mg/kg b.w. of Aredia™ on day 8.

Animals were sacrificed when symptoms caused by tumor growth appeared, i.e. when they developed hind-leg paralysis or became inactive with a kyfotic (hunchback) posture. Symptom free animals were followed for at least 50 days after the treatment. The significance of treatment response was related to symptom free survival and evaluated using the Wilcoxon rank sum test.

### Results

Dose survival plots for rats treated with ²²³Ra under therapy regimen [A] and [B] are presented in FIGS. 1 and 2 respectively. Control animals showed symptoms between 20 and 30 days after tumor cell inoculation. The increase in survival for animals injected with 100 kBq/kg and 300 kBq/kg relative to the control was significant at the 1% level. Also, when taken together, the increase in survival of the animals in the 100 kBq/kg and 300 kBq/kg groups was significant compared to the control group (P <0.05). Further, the 100 kBq/kg group had 36% (5/14) symptom free survivors beyond 50 days, while the animals injected with 60 kBq had 20% (1/5) symptom free survivors, but survival improvement was not significant since paralysis occurrences were not statistical significantly delayed compared to the control group.

Animals treated under regimen [B] are presented. The control group receiving Aredia™ had no survivors beyond day 21 and hence showed no response at the dose level administered. The 100 and the 300 kBq groups each had 40% (2/5) long term surivors. The 100 kBq and 300 kBq groups had a survival that was significantly improved (p <0.5) compared to the control. In Table 3 below the two different treatment regimens are compared. The data indicate survival benefit in the groups treated with Aredia and ²²³Ra compared to the groups treated with ²²³Ra alone. Long term survival (>67 days) was 40% in both Aredia plus ²²³Ra groups but reduced to 33% and 0% in the two groups treated with ²²³Ra alone. The mean survival was also slightly elevated in the ²²³Ra plus Aredia groups compared to the groups treated with ²²³Ra alone.

TABLE 3
Treatment efficacy against skeletal metastases in rats

| Without Aredia | | | | With Aredia | | |
|---|---|---|---|---|---|---|
| | Control | 100 kBq/kg | 300 kBq/ka | Control | 100 kBq/kg | 300 kBq/kg |
| Mean survival | 22 | 38 | 38 | 21 | 39 | 31 |
| (days) | | | | | | |
| Survival range | 20-29 | 20->67 | 27-61 | 20-21 | 21->67 | 21->67 |
| Survival beyond | 0% | 33% | 0% | 0% | 40% | 40% |
| 67 day | | | | | | |

What is claimed is:

1. A method of prophylaxis of a subject having or having had a soft tissue cancer, whereby to combat metastatic bone cancer therein, said method comprising administering to said subject a physiologically tolerable polyphosphonate and, parenterally, a prophylactically effective amount of a soluble radium-223 or -224 salt or complex.

2. The method of claim 1 wherein said subject is human.

3. The method or claim 1 wherein the radium complex is administered in a solution containing at least one other alkaline earth metal cation, at least one agent to prevent radium precipitation or colloid formation and/or at least one complexing agent.

4. The method of claim 3 wherein the alkaline earth metal cation is a physiologically tolerable calcium salt.

5. The method of claim 1 wherein the polyphosphonate is at least one selected from the group of bisphosphonates, tri-phosphonates, tetra-phosphonates and penta-phosphonates.

6. The method of claim 5 wherein the polyphosphonate is at least one bisphosphonate selected from the group of pamidronate, aledronate, ibandronate, zoledronate and risedronate

7. The method of claim 1 wherein the subject has been previously diagnosed with soft tissue cancer but is without any symptoms or clinical manifestations of metastatic bone disease.

8. The method of claim 7 wherein the subject has been previously diagnosed with breast, lung, kidney, urinary tract or prostate cancer or with multiple myeloma.

19. The method of claim 1 wherein the subject has cancer cells with at least one over or under expression selected from high levels of oestrogen receptors, high levels or androgen receptors, high levels of laminin receptors, high levels of the parathyroid hormone relate protein (PTHrP), or low levels of cell-adhesion proteins.

10. The method of claim 1 wherein each dose of radium is followed by at least one dose of polyphosphonate.

11. A kit comprising
(a) a first container containing a soluble radium-223 or -224 salt or complex and
(b) a second container containing at least one dose of a physiologically tolerable polyphosphonate.

12. The kit or claim 11 additionally comprising instructions for administration.

13. The kit of claim 11 additionally comprising means to encourage adherence to a pre-defined treatment regime.

14. The kit of claim 11 additionally comprising administration means.
